(19) United States
(12) Patent Application Publication
(10) Pub. No.: US 2009/0204105 A1
(43) Pub. Date: Aug. 13, 2009

(54) COUPLING DEVICE FOR MEDICAL PURPOSES AND AN ACCESS SYSTEM INCLUDING SUCH A COUPLING DEVICE

(75) Inventors: Roger Johansson, Knivsta (SE); Dick Persson, Los (SE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: GAMBRO CREDIT AB, STOCKHOLM (SE)

(21) Appl. No.: 12/161,138

(22) PCT Filed: Nov. 29, 2006
(86) PCT No.: PCT/SE06/01363

§ 371 Date: Nov. 12, 2008

(30) Foreign Application Priority Data
Jan. 17, 2006 (SE) 06000079-8

(51) Int. Cl. A61M 39/10; A61M 39/02

(52) U.S. Cl. 604/533

(57) ABSTRACT

A coupling device (3,4) for medical purposes with at least one coupling channel (9,18) having a mouth in a coupling surface (10), for sealed co-operation with at least one access channel (7) with a mouth in a connecting surface (β). of an access device (2), which is connectable to a patient, is distinguished in that the coupling surface (10) includes a flexible portion (11) for contacting against the connecting surface (6) and being of a material having a higher degree of flex than a primary material of the coupling device, which portion for sealing purposes surrounds the mouth of said at least one coupling channel (9,18).

### FIELD OF THE INVENTION

The invention concerns a coupling device according to the preamble of claim 1 and an access system including at least one such coupling device.

### BACKGROUND OF THE INVENTION

From the applicant's own WO 99/20338 is previously known coupling devices of this kind for connection to a blood access device. Each coupling device has at least one coupling channel, which is intended for a sealed connection to an access channel in the blood access device. The blood access device has a guide for coupling devices which is open at both ends. For the purpose of achieving a sealed connection, sealing surfaces on both elements co-operate with each other. Further there are means for pressing the sealing surfaces against each other for eliminating possible play and thus achieve a desired sealed connection.

### AIM AND MOST IMPORTANT FEATURES OF THE INVENTION

It is an aim of the invention to provide an improvement of the above coupling device that allows maintaining a secure seal between two components in an access system and this in respect of devices which can be produced to a reduced cost in comparison with the background art. This aim is obtained in a coupling device as above through the features of the characterizing portion of claim 1.

By the coupling surface, for contacting against the connecting surface, having a flexible portion which surrounds the mouth of the coupling channel/channels, increased security with respect of the sealing function is achieved. Further, an important advantage with the invention is that the requirements for strict manufacturing accuracy which exist in respect of devices according to the background art are not required to be met in devices according to the invention, which in turn results in simplified manufacture and thereby economic advantages in production.

It is important to note that the invention allows that the access device can be manufactured without previous strict requirements for dimensional tolerances, which allows production with a minimum of, or even completely eliminated subsequent finishing treatment.

By forming a rim-shaped seal, a part of the coupling surface formed in that fashion is said flexible portion, which allows simplified production of the coupling device according to the invention.

By the presence of a protruding sealing lip, the possibilities of taking up unevenness between the coupling surface and the connecting surface are enhanced in order to achieve seal between these surfaces.

By the flexible portion being attached in a groove, stability of the flexible portion is ensured, which is important, since positioning and exchange of coupling devices requires a translational movement and thus the occurrence of shearing forces.

By providing a space which is adapted to receive a part of the flexible portion after positioning thereof on an access device, it is allowed that the material in the flexible portion, when being pressed in the direction toward the coupling surface, can be simply transferred in directions at right angles to said pressing direction, which among other things allows that the flexible portion, when being positioned, will not exert excessive pressing forces against the surface of the access device.

By providing a sideward directed sealing portion, leakage is counter-acted in the region between two coupling devices in the process of exchanging coupling devices on an access device. This could otherwise be a problem, since during exchange of coupling devices according to the background art, a certain limited amount of for example blood leaks out, which can be experienced as unpleasant by patients and other people involved. In this connection it can also be mentioned that it is always an advantage to reduce the exposure of the surroundings to blood, in case of a blood access device, since blood can always be contaminated and thereby become a source of infection for the surroundings.

It is preferred that the sideward directed sealing portion is integral with the flexible portion, since this results in technical and economical advantages in production. The presence of a sideward directed sealing lip contributes to enhanced sealing in this area.

Coupling devices according to the invention can advantageously be used for different types of external equipment for the access device, such as a coupling unit for connection to an external circuit, to a fluid source, be a protective unit for an access device or a short-circuiting unit, wherein the latter allows a flow between two channels in an access device, as well as a penetrable lid, which can be penetrated for the insertion of different substances for medical purposes. Further, a coupling device according to the invention can have sensor means for monitoring a blood parameter such as a temperature or a concentration of a substance. The access device can be implanted or free-standing. It can be a blood access device or an access device for accessing other body organs than blood vessels.

The flexible portion can be produced from a plurality of suitable materials such as silicon rubber or any other rubber or rubber-like material, thermoplastic elastomer or be made of any other suitable elastic plastic.

The flexible portion and, occasionally, the sideward directed sealing portion is advantageously adhered to the primary material of the coupling device. This can be achieved through the manufacturing process of a coupling device, whereby adhesion can be obtained.

By providing the flexible portion peripherally of the coupling surface, the possibility is minimized for external particles to enter between a coupling device and an access device.

It is preferred that the flexible portion has a protrusion outside the coupling surface of about 0.01-0.5 mm for normal use. This gives a good combination of manufacturing friendliness and security of the sealing function as well as simplicity during application.

The invention also concerns an access system including a coupling device, whereby the advantages corresponding to the above are obtained.

### BRIEF DESCRIPTION OF DRAWINGS

The invention will now be described in greater detail by way of embodiments and with reference to the annexed drawings, wherein:

FIG. 1 shows parts of an access system including coupling devices according to the invention,

FIG. 2 shows an access device for the connection of a coupling device according to the invention,

FIG. 3 shows, partly in section, a coupling device according to the invention connected to an access device,

FIG. 4 shows in a perspective from below, a coupling device in a first embodiment,

FIG. 5 shows in a perspective from below, a coupling device according to a second embodiment, and

FIG. 6 shows a diagrammatical section through the region of a flexible portion.

### DESCRIPTION OF EMBODIMENTS

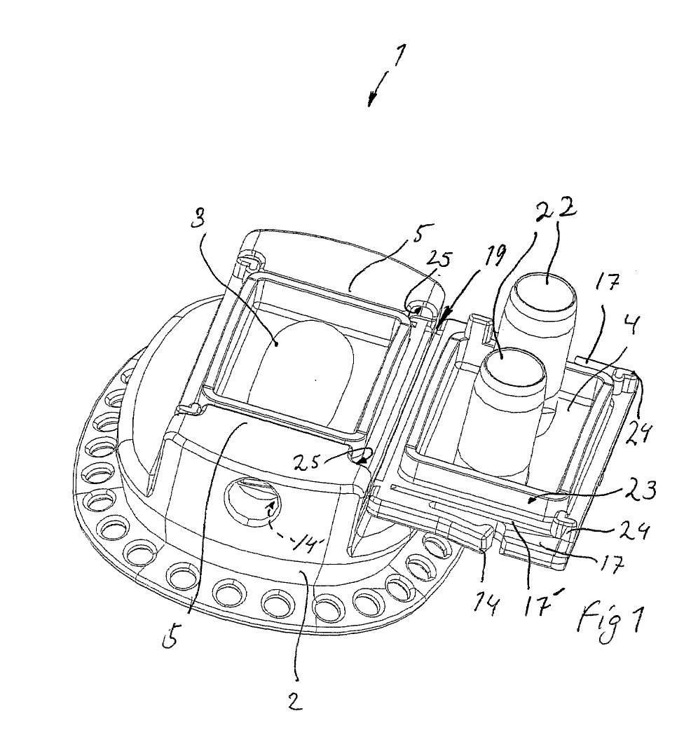

FIG. 1 shows an access system with the very application device, different connections etc. eliminated for the sake of clarity. On an access device 2 is held a coupling device 3 of a first embodiment as a short-circuiting unit. Sideward of this short-circuiting unit 3 is shown a coupling device 4 according to a second embodiment which is as a coupling unit having connections 22 to an external circuit.

For better understanding of the general purpose of an access device according to the invention reference is made to the applicant's own WO 99/20338, wherein also the principle of the function of an application device is described.

The access device 2 has first and second holding elements 5 for the co-operation with holding means 17 which are arranged transversally of the respective coupling devices 3 and 4, and which extends over the width of the side of the respective coupling device 3 and 4. The holding elements 5 form a guide for coupling devices that is open in both ends which is shown in more detail in said WO document. On the holding means 17, there are arranged strips 17′ (one shown in the figure) for vertical (as seen in the figure) positioning of the coupling device against the holding surface of the holding elements 5.

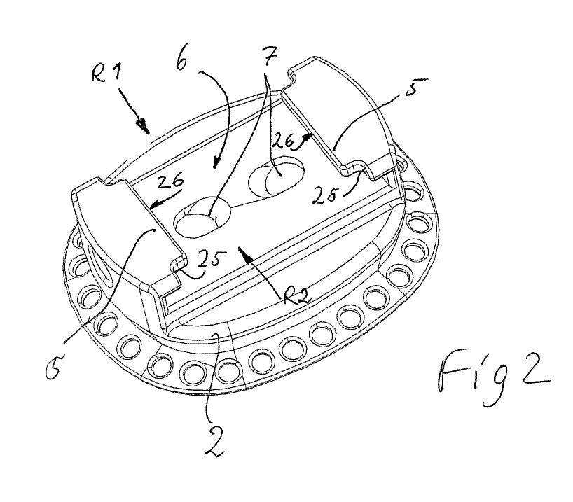

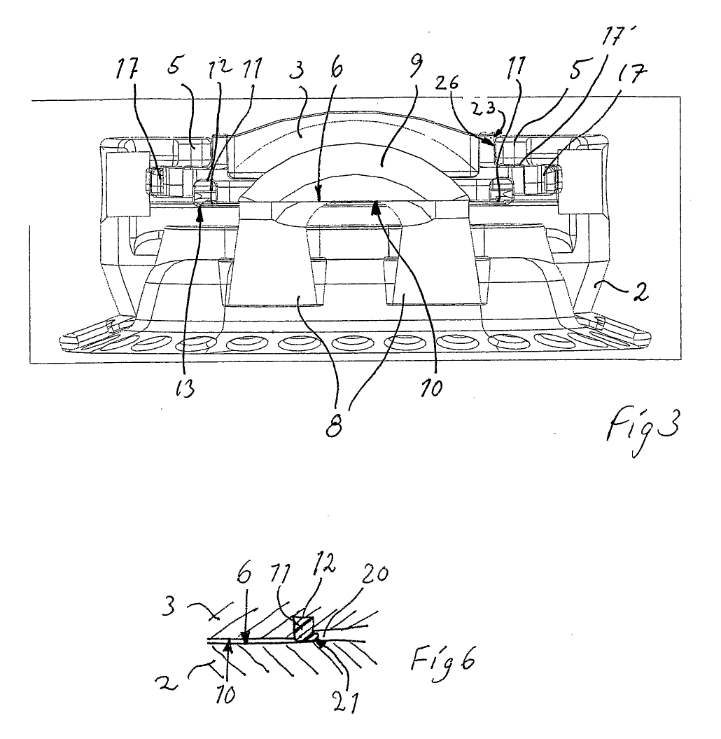

At an angle of a coupling surface (10 in FIGS. 3, 4 and 5), preferably at about right angle, extends on each side a positioning surface 23, which is arranged to co-operate with a positioning edge (26 in FIGS. 2 and 3) on the holding element 5 in order to achieve an adequate positioning in horizontal direction transversal to a direction of inserting the coupling device. For positioning in a direction of insertion are arranged firstly snap lock arms 14 on the coupling device 4, that co-operate with holding edges (indicated at 14′) on the access device 2. Secondly, the coupling device 4 has stop lugs 24, which cooperate with stop recesses 25 on the access device, at the ends of each holding element. This way, good precision during application is ensured, which increases the precision of the channel formed by the access device and the coupling device so that thereby protruding edges and recesses that negatively affect the flow because of erroneous positioning are avoided.

FIG. 2 shows the access device 2 in greater detail, whereby application directions for coupling devices are indicated with arrows R1 and R2, respectively. With 6 is indicated a connecting surface and with 7 two mouths to two access channels for the connection to a patient.

FIG. 3 shows in a section an access device 2 with positioned coupling device 3 of the kind that includes a short-circuiting channel 9. In the figure is shown that the holding elements 5 co-operate with the holding means 17 and in particular the strip 17′ for adequate positioning in a vertical direction as seen in the figure. With 6 is indicated the connecting surface of the access device and with 10 a coupling surface of the coupling device 3.

Outward of the short-circuiting channel 9 is positioned a surrounding groove 12 for receiving a strip-shaped means comprising a flexible portion 11, which is arranged with one part to protrude past, or outside the plane of said coupling surface 10. The flexible portion 11 comprises a sealing lip 13, which has a more limited, in the protrusion direction narrowing extension, which sealing lip functions well in order to receive possible tolerance deviations of the parts involved.

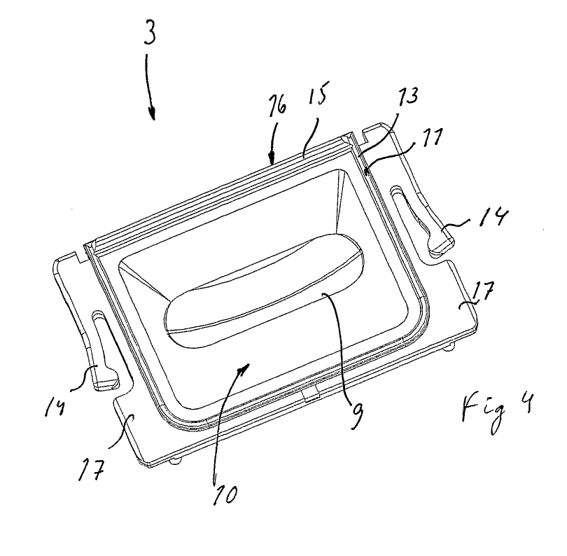

FIG. 4 shows the underside of the coupling device 3, wherein is shown the short-circuiting channel 9 which is positioned inside the surrounding strip-shaped flexible portion 11 with its sealing lip 13. Upwardly in the figure is directed a sideward directed sealing portion 15 with a sideward directed sealing lip 16, which has the purpose of sealingly contact against another coupling device during the process of exchanging coupling devices on an access device 2. Said sideward directed sealing portion 15 with the sealing lip 16 guarantees that leakage in the area of the arrow 19 in FIG. 1, i.e. between two contacting coupling devices, is avoided or at least reduced in relation to what is the case with the background art.

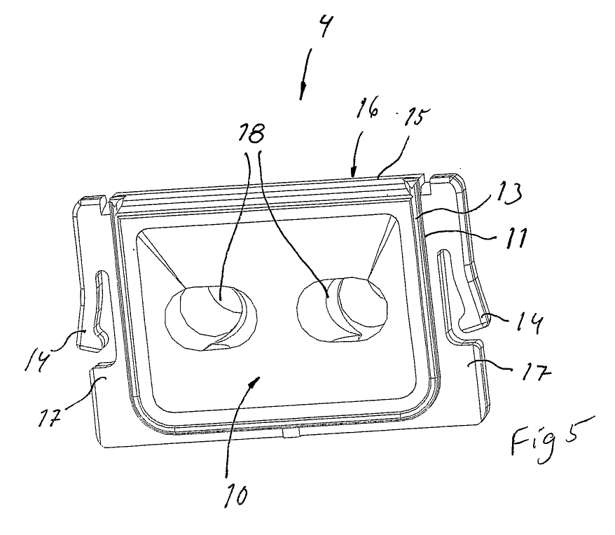

FIG. 5 differs from FIG. 4 only by the fact that this figure concerns a coupling device 4 of the kind which is a coupling unit as shown in FIG. 1. All details relating to the present invention are the same as is shown in FIG. 4. With 18 are indicated mouths of the coupling channels of the coupling device 4.

FIG. 6 shows a diagrammatical section through a flexible portion 11 which is positioned in a groove 12 of a coupling device 3. Contact between the coupling surface 10 and the connecting surface 6 results in an inward pressing of the flexible portion. This in turn results in that a part 21 of the flexible portion will be pressed out into a space 20 which is arranged in the vicinity thereof, since the flexible portion in principle is not compressible and an inward pressed volume must find space somewhere.

The invention can be modified within the scope of the following claims. Thus, the flexible portion can be shaped otherwise, and it is not excluded, for example, that as much as the entire coupling surface is covered with a flexible material even if such a solution is not preferred. In particular it is preferred that a strip-shaped portion is formed such that the material of the flexible portion, which extends outside the coupling surface, can be received in an adjacent space, either at the side of for example a groove for receiving the flexible portion in the form of a strip, or in such a way that inside the strip there is, on the one hand a protruding sealing lip, on the other hand a recess, with respect to the level of the coupling surface for receiving pressed-in flexible material.

The sideward directed sealing portion is preferably formed such that the space between the two contacting coupling devices is small, such that under the process of exchanging coupling devices on an access device, the amount of blood being present here is limited.

The term “coupling channel” should be interpreted to include all channels or parts thereof of a coupling device, firstly such having a mouth in a coupling surface, secondly short-circuiting channels leading a liquid from an access channel to another.

Coupling devices according to the invention can be of different kinds besides the ones mentioned above, thus a coupling unit to be connected to an external circuit can be used for extracorporeal blood treatment such as for example CAVH. A coupling unit for connecting to a fluid circuit source can be used for medication, supply of nutrition etc. Further, a coupling device according to the invention can include sensor elements, for example for temperature control, which can be used on the one hand for monitoring the function of the access device, on the other hand show certain conditions of the patient. Other parameters in the blood can be monitored, for example the concentration of different substances. This can be indicated with the aid of different kinds of signal systems.

The flexible portion and the sideward directed sealing portion can be produced from any material from the group silicon rubber or any other synthetic rubber or natural rubber, thermoplastic elastomeric material or any other suitable flexible plastic. The primary material of the coupling device can be any suitable relatively hard, per se known plastic.

1. Coupling device (3,4) for medical purposes with at least one coupling channel (9,18) having a mouth in a coupling surface (10), for sealed co-operation with at least one access channel (7) with a mouth in a connecting surface (6) of an access device (2), which is connectable to a patient, and which includes holding elements (5) for the cooperation with holding means (17) on the coupling device (3,4), wherein the holding elements form a guide for coupling devices that is open at two ends, characterized in that the coupling surface (10) includes a flexible portion (11) for contacting against the connecting surface (6) and being of a material having a higher degree of flex than a primary material of the coupling device, which portion for sealing purposes surrounds the mouth of said at least one coupling channel (9,18).

2. Coupling device according to claim 1, characterized in that the flexible portion (11) is strip-shaped.

3. Coupling device according to claim 1 or 2, characterized in that the flexible portion (11) comprises a protruding sealing lip (13).

4. Coupling device according to claim 1, 2 or 3, characterized in that the flexible portion (11) is attached in a groove which is made in the coupling surface (10) of said primary material.

5. Coupling device according to any of the previous claims, characterized in that a space (20) is arranged in the vicinity of the flexible portion (11) in order to receive at least a part (21) of a portion thereof which protrudes outside the coupling surface (10) after the application on an access device (2).

6. Coupling device according to any of the previous claims, characterized by a sideward directed sealing portion (15) for contacting against a second coupling device in a process for application or exchange of coupling device on the access device (2).

7. Coupling device according to claim 6, characterized in that the sideward directed sealing portion (15) is integral with the flexible portion (11).

8. Coupling device according to claim 6 or 7, characterized in that a sideward directed sealing portion (15) has a sideward directed sealing lip (16).

9. Coupling device according to any of the previous claims, characterized in that it is any from the group: a coupling unit for connection to an external circuit (4), a coupling unit for connection to a fluid source, a protective unit, a short-circuiting unit (3), a penetrable lid.

10. Coupling device according to any of the previous claims, characterized in that the flexible portion (11) and at occurrences the sideward directed sealing portion (15) is or are manufactured from any material from the group: silicon rubber, thermoplastic elastomer, rubber, elastic plastic.

11. Coupling device according to any of the previous claims, characterized in that the flexible portion and at occurrences the sideward directed sealing portion are adhered to the primary material of the coupling device.

12. Coupling device according to any of the previous claims, characterized in that the flexible portion is arranged peripherally on the coupling surface.

13. Coupling device according to any of the previous claims, characterized in that flexible portion has a protrusion outside the coupling surface of about 0.01-0.5 mm.

14. Access system for the connection to a patient for medical purposes, including an access device (2), an application device for coupling devices and at least one coupling device (3,4) according to any of the claims 1-13.
